(19) United States
(12) Patent Application Publication
(10) Pub. No.: US 2004/0230286 A1
(43) Pub. Date: Nov. 18, 2004

(54) Stent introducer apparatus

(76) Inventors: Scott T. Moore, Rural Hall, NC (US); Victor D. Clark, Pfafftown, NC (US)

Correspondence Address: Lawrence G. Almeda, Esq., BRINKS HOFER GILSON & LIONE, P.O. Box 10395, Chicago, IL, 60610, US

(21) Appl. No.: 10/796,215

(22) Filed: Mar. 9, 2004

Related U.S. Application Data
(60) Provisional application No. 60/453,374, filed on Mar. 10, 2003.

(51) Int. Cl. A61F 2/06

(52) U.S. Cl. 623/1.11

(57) ABSTRACT

A stent introducer apparatus comprising an introducer catheter and a pusher assembly configured to deliver a stent, such as a self-expanding stent, within a tortuous duct or vessel, even if the pusher member is acutely positioned during deployment. The pusher assembly includes a first tubular portion comprising a material with high column strength and a shorter second tubular portion made of a highly flexible material. The second tubular portion is divided into a distal, stent-carrying section and a proximal, flexible section. The second tubular portion may be made of a smaller diameter than the first tubular portion to reduce possible impingement by the introducer catheter is the latter kinks during a procedure. A soft pusher member is disposed at location between the stent-carrying and flexible sections to absorb preload pressure and to urge the stent from the distal end of the introducer catheter.

### CROSS-REFERENCE TO RELATED APPLICATIONS

This application claims priority to U.S. Provisional Application No. 60/453,374, filed Mar. 10, 2003, entitled “Stent Introducer Apparatus.”

### FIELD OF THE INVENTION

This invention relates to medical devices. More particularly, the present invention relates to an apparatus for delivering an implantable prosthesis.

### BACKGROUND OF THE INVENTION

Placement of a stent within the biliary tree can be problematic, since a catheter delivery system must make a severe turn from the duodenum through the opening of the common bile duct. Current biliary and pancreatic stent delivery systems comprise an introducer catheter with a stent loaded at the distal end of the introducer catheter. A pusher catheter is used to deploy the stent from the introducer catheter. Physicians prefer that the introducer catheter be made of a clear material so that they can see the stent within the catheter. This usually requires that the catheter be made of a plastic material which, by the nature of the material, makes the catheter prone to kinking. When the introducer catheter kinks, it can impinge on the pusher catheter and prevent the stent from being deployed. While the stent and pusher catheter serve to fill the lumen of the introducer catheter, making kinking within these portions less of a problem, the junction between the stent and pusher is a vulnerable point on the catheter where a kink can occur. If a kink does occur, the pusher may not be able to traverse the catheter stricture to advance the stent.

Some manufacturers have attempted to avoid this problem by using an axially contracting stent which overlaps the distal end of the pusher. As a result, the most juncture between the stent and the pusher (the most likely kinking point) is reinforced by a portion of the stent. However, this system has other disadvantages. In particular stents that shorten (i.e., axially contracting stents) are less desirable than non-contracting stents because of difficulty in their placement. Non-shortening biliary stents, such as the ZA-STENT™ or SPIRAL Z™ Biliary Stents (Wilson-Cook Medical, Inc., Winston-Salem, N.C.), can be placed more accurately and provide superior coverage. However, the point on the catheter most susceptible to kinking is not reinforced by the stent, making kinking more of a concern when polytetrafluoroethylene (PTFE) is used for the introducer catheter.

Another common problem with current biliary stent delivery systems is diminished recapture capability. Manufacturers have been challenged in providing the ability to retrieve the introducer system following stent delivery without having the introducer system become caught within the stent or upon the introducer catheter itself.

It has also been determined that undesired partial deployment of the stent may occur when the pusher junction, the junction between the stent and the pusher, is positioned at an acute bend in the body during deployment of the stent. This bending may cause inadvertent displacement of the stent relative to the pusher. As a result, deployment of the stent may be limited only to segments in the body which are free of acute bends.

What is needed is a biliary and pancreatic stent introducer system that allows deployment of the stent independent of an acute position of the pusher junction during deployment of the stent. What is further needed is a biliary and pancreatic stent introducer system that reduces kinking at the pusher junction. What is also needed is a biliary pancreatic stent introducer system that can still be deployed when the outer catheter kinks and easily removed once the stent is deployed.

### SUMMARY OF THE INVENTION

The foregoing problems are solved and a technical advance is achieved in a stent introducer apparatus having a pusher assembly with a lumen therethrough for introduction of a wire guide. The pusher assembly can be used to deploy a preloaded self-expanding stent from the distal end of an introducer catheter, such as a PTFE introducer sheath configured for use in the biliary or pancreatic ducts. The pusher assembly comprises a first or proximal tubular portion that substantially fills the introducer catheter lumen and is made of a material with superior column strength, such as polyetheretherketone (PEEK), and a second or distal tubular portion which has a combination of good column strength and superior flexural properties, such as braided polyimide or nitinol, to distribute any bending forces more evenly along the introducer catheter and help reduce the severity of kinking. A pusher member is disposed in and along the second tubular portion of the pusher assembly. The pusher member is designed to absorb preload pressure and to urge the stent forward. The pusher member conforms to the proximal end of the stent when the stent is preloaded in the introducer catheter so as to prevent undesired partial deployment. The pusher member can comprise one or more separate elements attached to the second tubular portion or it can be an integral modification thereof that provides a mechanism for advancing or deploying the stent.

In one embodiment, at least a part of the second tubular portion distally extends from the first tubular portion. The second tubular portion includes a flexible section and stent-carrying section located distally from the flexible section. In this embodiment, the pusher member is a soft pusher member configured to urge the preloaded stent from the introducer catheter. The soft pusher member is disposed along the second tubular portion at a point which is either proximal to or within the stent-carrying section. In this embodiment, the soft pusher member is made of a polymer having a radiopaque filler and is configured to cooperate with the preloaded stent for absorbing preload pressure or force of the preloaded stent when the soft pusher member is positioned at an acute bend in the body of a patient. Cooperation between the soft pusher member and the preloaded stent reduces kinking at the juncture point between the stent and the soft pusher member, particularly when the soft pusher member is at an acute bend of the body during deployment of the stent.

In another embodiment, the pusher member comprises a pusher head made of metal or an insert-molded polymer that provides a broad surface for applying force to advance the stent. The stent is loaded while applying pressure against the pusher head to reduce any gap therebetween. This helps to direct any kinks that may be experienced during the procedure to occur proximal to the pusher member, thereby not interfering with the ability of the pusher assembly to advance the stent from the introducer catheter.

In another aspect of the invention, the pusher member is configured such that the proximal portion of the pusher member can more easily negotiate a kink in the introducer catheter during withdrawal of the pusher assembly following delivery. This can be accomplished by tapering the distal tubular portion. In this embodiment, a similar proximal taper occurs on the distal tip of the pusher assembly, located distal to the stent. The face of the pusher member contains a chamfer to help prevent it from digging into the inner wall of the introducer catheter. Moreover, there is a second member at the junction between the second tubular portion and the first tubular portion. The second member is tapered distally to help facilitate its advancement through any kink that might occur along the section of the introducer catheter that is distal to that point.

Further objects, features and advantages of the invention will become apparent from consideration of the following description and the appended claims when taken in connection with the accompanying drawings.

### BRIEF DESCRIPTION OF THE DRAWINGS

FIG. 1 is a partially sectioned side view of a stent introducer apparatus in accordance with one embodiment of the present invention;

FIG. 2 is an enlarged sectional view of a portion of the stent introducer apparatus of FIG. 1;

FIG. 3 is a partially sectioned view of the stent introducer apparatus of FIG. 1 in a kinked introducer catheter;

FIG. 4 is a partially sectioned view of a pusher member in accordance with another embodiment of the present invention;

FIG. 5 is a sectional view of one embodiment of the present invention in which a second tubular portion extends at least substantially the length of a first tubular portion;

FIG. 6 is a sectional view of one embodiment of the present invention in which the first and second tubular portions of a pusher assembly include a single member; and

FIG. 7 is a sectional view of another embodiment of the present invention in which the first and second tubular portions of the pusher assembly comprise a single member.

### DETAILED DESCRIPTION

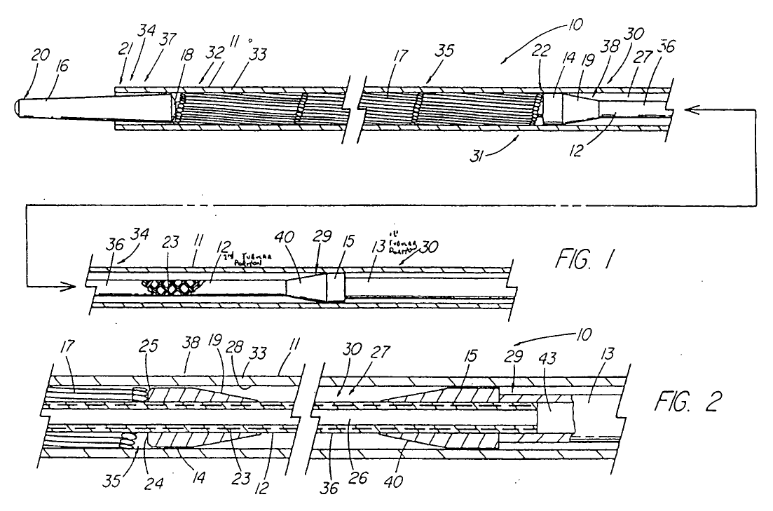

FIGS. 1 and 2 illustrate a stent introducer apparatus 10 in accordance with one embodiment of the present invention. As shown, the stent introducer apparatus 10 comprises a pusher assembly 30 for advancing a stent 17 for deployment within a duct or vessel. In the embodiment shown in FIG. 1, the stent is a self-expanding biliary stent such as the COOK SPIRAL Z™ Stent or the ZILVER™ stent. However, the type of stent is not considered important to the understanding of the invention. In this embodiment, the minimum size of the introducer catheter typically ranges from 7.0 to 8.5 FR (2.33 TO 2.83 mm), depending on the stent used. The SPIRAL Z™ biliary Stent, being somewhat larger than the ZA-STENT™ Biliary Stent, requires the larger introducer, while the smaller stent can be deployed from either sized introducer.

As depicted in FIGS. 1 and 2, the stent introducer apparatus 10 may further include an introducer sheath or catheter 11 which, in this embodiment, is made primarily of a substantially clear polymer such as polytetrafluorothylene (PTFE). The pusher assembly 30 and the preloaded stent 17 are coaxially disposed within passageway 27 of the introducer catheter 11. The stent 17 resides in the distal portion 34 of the introducer catheter until it is expelled from the distal end 21 thereof by advancement of the pusher assembly 30 and/or withdrawal of the introducer catheter 11.

The pusher assembly of FIGS. 1 and 2 comprises a first or proximal tubular portion 13 and a second or distal tubular portion 12. The first and second tubular portions 12, 13 can be formed as separate members and attached together, or comprise different portions of a single member. As will be explained in more detail below, the portions 12, 13 have different physical properties. Each portion 12, 13 has a lumen formed therethrough that is sufficiently large for accommodating an ancillary device such as a 0.035″ (0.89 mm) wire guide. The first tubular portion 13 can comprise a rigid or non-rigid member or portion thereof, depending on the application. In this embodiment, the first tubular portion 13 comprises a non-rigid polymer tube made of a material with superior column strength. Possible materials include, but are not limited to, PEEK, polyvinyl chloride (PVC), polyimide, and polyurethane. The outside diameter (O.D.) of the first tubular portion 13 is approximately 0.07″ (1.78 mm) in this example, and is configured to take up most of the inside diameter (I.D.) of the passageway 27 of the introducer catheter 11 so as to provide support thereto and reduce the likelihood and severity of kinking in the introducer catheter 11. Maximizing the pusher catheter O.D. also adds rigidity and column strength for pushing the stent from the catheter.

The second tubular portion 12 extends distally from the first tubular portion 13 to which it is joined. The second tubular portion 12 comprises a tube made of a flexible material with sufficient column strength to allow the pusher assembly 30 to advance the stent from the introducer catheter 11. In this embodiment, the second tubular portion 12 comprises a polyimide tube reinforced with a stainless steel braid. Other possible materials include PEEK or metal tubing such as nitinol or stainless steel, depending on the degree of bending that the introducer is anticipated to undergo. Nitinol tubing exhibits adequate lateral flexibility and kink-resistance, but is generally more stiff than braided polyimide tubing. Both the pusher assembly 30 and the introducer catheter 11 are connected at their proximal ends to a well-known coaxial medical device handle (not illustrated) that permits the pusher assembly 30 to be advanced relative to the introducer catheter 11 for deployment of the stent 17. An example of a suitable slider-type handle can be found on the previous-generation delivery systems for the Wilson-Cook SPIRAL Z™ and ZA-STENT™ Biliary Stents.

In this embodiment, a pusher member 14 is affixed to or integrally formed with the second tubular portion 12 to receive preload pressure of the stent 17 and to push the stent 17 out of the introducer catheter. In this embodiment, the pusher member 14 comprises a pusher head. The pusher head includes a broad face 24 to contact and conform with the proximal end 31 of the stent 17 and urge the stent forward until deployment has been achieved.

In one embodiment, the illustrative pusher member 14 is a relatively soft pusher member made of a low density polymer having a radiopaque filler. However, many other suitable types of material may be used. The pusher member may be insert-molded, bonded, or otherwise attached to the second tubular portion. In another embodiment, the pusher may be made of metal such as 303 or 304 stainless steel.

The O.D. of the pusher member generally depends on the type of stent to be delivered. In this embodiment, a SPIRAL Z™ Biliary Stent, which is deliverable through an 8.5 Fr (2.83 mm) introducer catheter, would have a 0.088″ (2.24 mm) O.D. pusher member 14. The ZA-STENT™ Biliary Stent, which is can be introduced through either an 8.0 or 8.5 Fr (2.67 or 2.83 mm) introducer, could have a 0.077″ O.D. (1.96 mm) pusher member 14 if the 8.0 Fr (2.67 mm) introducer is used. The dimensions of the pusher member 14 could vary further, depending on a number of factors, particularly the I.D. of the introducer catheter lumen 27. Because of the desirability of having the pusher member 14 diameter be as close to the I.D. of the introducer catheter lumen 27 as possible, an optional chamfer 25 is included at the outside edge of the face 24 to help prevent the pusher member 14 from digging into the inner wall 28 of the introducer catheter 11 during advancement.

In this embodiment, the pusher member 14, is placed over and glued to the second tubular portion 12 such that the contact point 22 between the two lies at an intermediate point along the second tubular portion 12. As shown, the pusher member is located along the second tubular portion at a point that is either proximal to or within the stent-carrying section. The pusher member cooperates with the preloaded stent to absorb preload pressure or force of the preloaded stent. This is useful particularly when the pusher assembly and the preloaded stent are disposed within the introducer catheter and when the pusher member is located in an acute bend in the body of the patient. It has been determined that cooperation between the stent and the pusher member allows the pusher member to receive the stent and absorb preload pressure thereof. This avoids an undesirable partial deployment of the stent and prevents a kink of the introducer catheter at an acute bend in the body.

In the illustrative embodiment, the pusher member 14 represents a junction 38 between two sections of the second tubular portion 12. The flexible section 36 of the second tubular portion 12 lies proximal to the pusher member 14. The stent-carrying section 35 of the second tubular portion 12 lies distal to the contact point 22 of the pusher member 14. While these two sections 35, 36 comprise a single piece of reinforced polyimide tubing in the illustrative embodiment, it is also possible that they be constructed with different materials or properties such that each section 35, 36 is likely to experience bend stresses during introduction due to the presence of the preloaded stent 17 over the stent loading section 35. The length of the stent loading section 35 corresponds to the length of the stent 17.

A distal tip 16, made of PEBAX® (Atofina Chemicals, Philadelphia, Pa.) or any other suitable polymer having appropriate bonding properties, is bonded to the distal end 37 of the second tubular portion 12 after the stent 17 has been preloaded thereon. The distal tip 16 may include barium sulfate or some other agent or marker to provide radiopacity. Both the distal tip 16 and distal end 21 of the catheter are rounded for atraumatic entry into the bile duct.

The pusher assembly 30 provides an advantageous combination of both strength and flexibility that is desirable for biliary access. The section of the second tubular portion 12 proximal to the contact point 22 provides the stent introducer apparatus 10 with the ability to make a tortuous bend, such as into the opening of the common bile duct, by distributing the bending stresses over a large area (approximately 20 cm in the illustrative embodiment). In the illustrative embodiment, the second tubular portion 12 is made to have a smaller O.D., approximately 0.045″ (1.14 mm), to increase lateral flexibility. The first tubular portion 13 comprises the majority of the pusher assembly 30 because of the increased column strength and protection to the introducer catheter 11 it provides.

For example, a pusher assembly 30 might measure 190 cm from the proximal end of the catheter (distal end of the handle) to the proximal end 31 of the stent 17, wherein 160 cm of this length might comprise the first tubular portion 12 with only 30 cm comprising the flexible section 36 of the second tubular portion 12. Generally, the flexible section should comprise about 10% to 20% of the pusher assembly 30 in biliary applications. For other applications, the actual length of the flexible section can vary, depending on the application. For example, the entire stent introducer apparatus 10 could be made smaller for deploying vascular stents, or it could have utility in placing colonic stents where the anatomy can also produce a severe angle that can be of concern. For biliary applications, the distance from the junction between the handle and catheter to the distal end 20 of the introducer apparatus should generally measure at least 200 cm for a typical adult patient.

As shown in FIG. 2, the second tubular portion 12 is attached to the first tubular portion 13 by a well-known bonding method such as gluing. In this embodiment, a second member 15 is placed at the junction 29 between the distal and first tubular portions 12, 13 and glued in place with the two portions overlapping each other by approximately 3-5 mm. The second member 15 is made of metal or plastic and may be a band similar to pusher member 14.

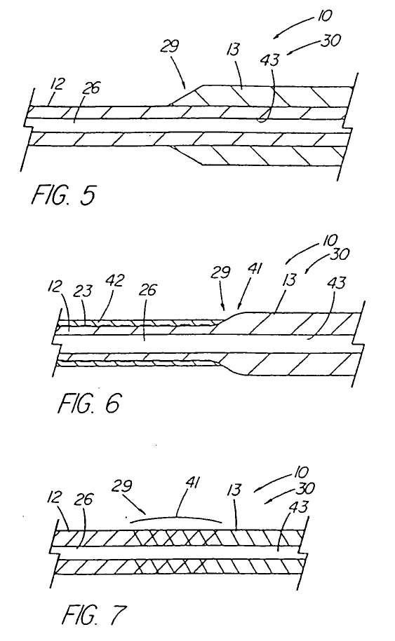

FIG. 5 depicts an embodiment in which the second tubular portion 12 extends the entire length (or nearly the entire length) of the first tubular portion 13. In this embodiment, the second tubular portion provides column strength and kink resistance (especially because of the increased diameter) to the proximal or remaining portion of the pusher assembly 12 proximal to initial junction 29 point. The second tubular portion 12 can be bonded along the length of the first tubular portion 13 or affixed at one or more points, such as junction 29.

FIGS. 6 and 7 depict additional embodiments of the pusher assembly 30 comprising a single continuous piece of tubing modified to produce a more flexible second tubular portion 12 and a more kink-resistant first tubular portion 13. The embodiment of FIG. 6 depicts a single-piece tube in which the first tubular portion 13 is smaller in diameter to form a thinner wall and a more flexible first tubular portion 12. Extrusion techniques to vary the diameter of thermoplastic tubing are well known in the catheter arts. In the illustrative embodiment, an optional braid 23 is added to the second tubular portion 12 to allow it to be more flexible and less prone to kinking. An optional second member 15, such as that of FIG. 1, can be affixed over a transition zone 41 (or junction 29) between the two tubular portions 12, 13 to facilitate negotiation of any kinks in the introducer catheter 11 that might form distal to that point. A thin layer 42 of polymer such as a shrink wrap or other type of polymer film can be added to secure the braided portion 42 to the outer surface of the second tubular portion 12.

In another embodiment, FIG. 7 depicts a pusher assembly 30 that has been extruded as two materials having different physical properties, including different degrees of column strength and flexibility. A first material, comprising the first tubular portion 13, blends with a second material comprising the second tubular portion 12 over a transition zone 41 from which the second tubular portion 12 extends distally. The second tubular portion 12 is generally more flexible than the proximal first tubular portion 13. The two materials are compatible for co-extrusion and can include different polymers or two different compounds (e.g., different durometers) of the same polymer. Methods of co-extruding different polymers to form a single length of tubing are well known in the catheter arts.

In assembling the illustrative stent introducer apparatus 10, the stent is loaded over the distal end 37 of the second tubular portion 12, and then distal tip 16 is placed thereover and bonded thereto, to hold the stent 17 in place. While the distal tip 16 is being affixed to the pusher assembly 30, pressure is applied such that the proximal end 31 of the stent 17 is forced tightly against the face 24 of the pusher member 14. This virtually eliminates any gap at the contact point 22, a gap which otherwise becomes a likely point of kinking when the introducer catheter is navigated through a severe bend, such as the common bile duct. The kink 39 generally occurs at that point along the introducer catheter 11 which experiences the greatest lateral bending forces during severe bending. This is largely determined by the degree of support provided by indwelling devices such as the pusher assembly 30 and the stent 17 itself.

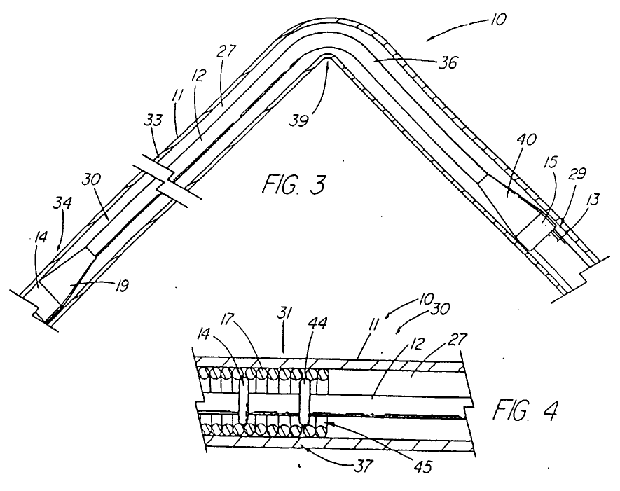

By reducing the weakness at the contact point 22 between the pusher member 14 and the stent 17, the most likely location of any kink 39 (see FIG. 3) in the introducer catheter 11 will be the flexible section 36 of the second tubular portion 12 which lies between junction 29 and the proximal end 31 of the stent 17. If a kink 39 develops within that section, it generally does not interfere with the ability of the pusher assembly 30 to slide within the introducer catheter 11 and expel the stent 17 therefrom. This is due to the pusher member 14 being distal to the kink 39, and in the case of the illustrative embodiment, the second tubular portion 12 is of a sufficiently small diameter such that the restriction of the introducer catheter lumen 27 still permits movement therethrough. Because this particular section of the introducer catheter 30 is flexible over an extended portion, any kink 39 that might occur is usually less severe than would be experienced in delivery systems of designs where the pusher system is stiff in comparison, and most of the bending force would be thus concentrated at the vulnerable contact point between the stent and the pusher member.

By absorbing preload pressure or force of the stent at the contact point, an undesirable partial deployment of the stent is avoided during the deployment of the stent. Partial deployment is mainly avoided or at least reduced when the soft pusher member is positioned at acute bends in the body during deployment of the stent. The soft pusher member receives preload pressure of the stent and conforms to the distal end of the stent when the pusher member is positioned at acute bends in the body during deployment.

The stent introducer apparatus 10 of FIGS. 1 and 2 is designed to facilitate recapture, i.e., removal of the pusher assembly 30 back through the deployed stent. A number of components or structures on a typical introducer apparatus have the potential of snagging and catching a strut, or otherwise becoming ensnared in the stent after delivery. To reduce the possibility of this occurring in the present invention, the proximal surface 18 includes a taper 18 that has been added to the distal tip 16 of the stent pusher assembly 30. In addition, proximal surface 19 of the pusher member 14 is tapered as well. These tapers reduce the likelihood of an edge catching the stent during withdrawal, and in particular, where the introducer catheter 11 is advanced by the physician after deployment to “recapture” the pusher assembly 30. The tapers 18, 19 also help guide the introducer catheter 11 over the distal tip 16 and pusher member 14 rather than having the distal end 21 of the introducer catheter 11 become temporarily caught. In addition, the proximal tapers 16, 18, especially that of the pusher member 14, help provide a guide to traverse any strictures during withdrawal of the pusher assembly 30 if the introducer catheter 11 becomes kinked.

It should be understood that the invention may include other shapes or modifications of the proximal surfaces 18, 19 of the distal tip and pusher member, other than a simple taper to provide a surface or edge that has a reduced likelihood or catching on the stent.

While the illustrative embodiment includes an expandable stent such as the SPIRAL Z™ Biliary Stent, knowledge of the type of stent to be used with the present invention, or how it is delivered is not essential for an understanding of the invention. Although the illustrative embodiment depicts a pusher member 14 to absorb preload pressure and to urge the stent 17 from the introducer catheter 11, alternative embodiments of the present invention may include a modified pusher assembly 30 that engages with the stent in another manner rather than pushing against the proximal end 31 of the stent 17.

For example, the second tubular portion could extend into the lumen of the loaded stent and be frictionally engaged therewith. For example, FIG. 4 depicts a second embodiment of pusher member 14 that urges the stent 17 forward by engaging the struts or coils of the stent 17 from inside the stent lumen 45 via one or more engagement members 44 affixed over the shaft of the second tubular member 12. These engagement members can be made of plastic or metal and vary in shape, number, and distribution along the stent loading portion 35 of the second tubular portion 12. When the stent 17 is deployed and expands, the engagement members 44 no longer engage the stent 17, permitting withdrawal of the pusher member 30.

Other embodiments could include a releasable engagement mechanism between the pusher assembly 30 and stent 17. Because of the variety of medical procedures for which this invention can be used, as well as the wide variety of stents that can be deployed, further modifications of the stent introducer apparatus of the present invention additional to the embodiments described herein are within the spirit of the invention and the scope of the claims. The invention contemplates embodiments comprising and consisting of the disclosed examples.

While the present invention has been described in terms of preferred embodiments, it will be understood, of course, that the invention is not limited thereto since modifications may be made to those skilled in the art, particularly in light of the foregoing teachings.

What is claimed is:

1. A pusher assembly of a stent delivery system for use in a target duct or vessel having an acute bend at a known general location in the body of a patient, the pusher assembly comprising:
a first tubular portion;
a second tubular portion having a part extending distally from the first tubular portion, the second tubular portion including a flexible section and a stent-carrying section located distally to the flexible section; and
a soft pusher member configured to urge a self-expanding preloaded stent from an introducer catheter within which the preloaded stent is slidably disposed, the soft pusher member being disposed along the second tubular portion at a location which is either proximal to or within the stent-carrying section, the soft pusher member being made of a polymer and cooperating with the preloaded stent to absorb preload pressure of the preloaded stent when the soft pusher member is positioned at the acute bend in the body during deployment of the preloaded stent.

2. The assembly of claim 1 wherein the flexible section of the second tubular portion has a preselected length and a location along the pusher assembly such that when the pusher assembly and the preloaded stent are disposed within the introducer catheter and are subjected to lateral bending stresses at the known general location in the body, the flexible section of the second tubular portion traverses the known general location in the body, whereby the likelihood of a kink occurring in the introducer catheter is greatest within a region corresponding to the region of greatest flexibility of the pusher assembly.

3. The assembly of claim 1 wherein the soft pusher member includes a radiopaque filler.

4. The assembly of claim 1 wherein the polymer of the soft pusher member is a low density polymer.

5. The assembly of claim 1 wherein the polymer of the soft pusher member is polytetrafluoroethylene.

6. The assembly of claim 1 wherein the second tubular portion has a smaller outer diameter than that of the first tubular portion.

7. The assembly of claim 6 wherein the second tubular portion comprises a metal-reinforced polymer material.

8. The assembly of claim 7 wherein the metal-reinforced polymer material comprises braided polyimide tubing.

9. The assembly of claim 1 wherein the second tubular portion comprises a nickel-titanium alloy.

10. The assembly of claim 1 wherein the second tubular portion extends to a distal end, the second tubular portion includes a distal tip affixed about the distal end, the stent-carrying section and the flexible section being comprised of a single continuous element, the preloaded stent being positioned along the stent-carrying section such that the preloaded stent is disposed between and in contact with the distal tip and the soft pusher member, the distal tip being tapered at its proximal end to receive the stent.

11. A stent delivery system for use in target duct or vessel having an acute bend at a known general location in the body of a patient, the system comprising:
a pusher assembly including a soft pusher member configured to urge a preloaded stent from an introducer catheter within which the preloaded stent is slidably disposed, the pusher assembly comprising a first tubular portion and a second tubular portion, at least a part of the second tubular portion extending distally from the first tubular portion, the second tubular portion including a flexible section and a stent-carrying section located distal to the flexible section, the soft pusher member being disposed along the second tubular portion at a location which is either proximal to or within the stent-carrying section, the soft pusher member being made of a low density polymer and configured to cooperate with the preloaded stent for absorbing preload pressure of the preloaded stent when the soft pusher member is positioned at the acute bend during deployment of the stent.

12. The stent delivery system of claim 11 wherein the flexible section of the second tubular portion has a preselected length and a location along the pusher assembly such that when the pusher assembly and the preloaded stent are disposed within the introducer catheter and are subjected to lateral bending stresses at the known general location in the body, the flexible section of the second tubular portion traverses the known general location in the body, whereby the likelihood of a kink occurring in the introducer catheter is greatest within a region corresponding to the region of greatest flexibility of the pusher assembly.

13. The stent delivery system of claim 11 wherein the soft pusher member includes a radiopaque filler.

14. The stent delivery system of claim 11 further including the stent preloaded within a distal portion of the introducer catheter, the stent having a proximal end and a distal end opposite the proximal end, the proximal end being received by the soft pusher member to absorb preload pressure of the stent.

15. The stent delivery system of claim 14 wherein the soft pusher member includes a face having a diameter equal to or greater than that of the preloaded stent while the stent is loaded in the introducer catheter, the proximal end of the preloaded stent being adjacent to the face of the pusher member.

16. The stent delivery system of claim 14 wherein the stent is a self-expanding stent.

17. The stent delivery system of claim 14 further including the introducer catheter in which the pusher assembly and the stent are slidably disposed.

18. The stent delivery system of claim 11 wherein the second tubular portion has a smaller outer diameter than that of the first tubular portion.

19. The stent delivery system of claim 18 wherein the second tubular portion comprises a metal-reinforced polymer material.

20. The stent delivery system of claim 19 wherein the metal-reinforced polymer material comprises braided polyimide tubing.

21. The stent delivery system of claim 11 wherein the second tubular portion comprises a nickel-titanium alloy.

22. The stent delivery system of claim 11 wherein the second tubular portion extends to a distal end, the second tubular portion includes a distal tip affixed about the distal end, the stent-carrying section and the flexible section being comprised of a single continuous element, the preloaded stent being positioned along the stent-carrying section such that the preloaded stent is disposed between and in contact with the distal tip and the soft pusher member, the distal tip being tapered at its proximal end to receive the stent.

23. A stent delivery system, comprising:
an introducer catheter having a distal portion, the distal portion having a distal end;
a stent preloaded within the distal portion of the introducer catheter, the stent having a proximal end and a distal end;
a pusher assembly including a soft pusher member configured to urge the preloaded stent from the introducer catheter within which the preloaded stent is slidably disposed, the pusher assembly comprising a first tubular portion and a second tubular portion, at least a part of the second tubular portion extending distally from the first tubular portion, the second tubular portion including a flexible section and a stent-carrying section located distal to the flexible section, the soft pusher member being disposed along the second tubular portion at a location which is either proximal to or within the stent-carrying section, the soft pusher member including a face having a diameter equal to or greater than that of the stent preloaded in the introducer catheter, the soft pusher member being made of a polymer and configured to cooperate with the preloaded stent such that the face of the soft pushing member absorbs preload pressure of the preloaded stent when the soft pusher member is positioned at the acute bend during deployment of the stent; and
a distal tip affixed about the distal end of the second tubular portion, the stent being loaded between the distal tip and the face of the pusher member such that during deflection of the stent introducer apparatus, the point along the introducer catheter that receives the largest amount of bending stress and represents the more likely point where a kink would occur, is located proximal the pusher member.

24. The stent delivery system of claim 23 wherein the second tubular portion comprises a metal-reinforced polymer material.

25. The stent delivery system of claim 24 wherein the metal-reinforced polymer material comprises braided polyimide tubing.

26. The stent delivery system of claim 23 wherein the second tubular portion comprises a nickel-titanium alloy.

27. The stent delivery system of claim 23 wherein the second tubular portion further includes a stent-carrying section extending distal the flexible section, the stent-carrying section extending distally to at least the distal end of the stent.

28. The stent delivery system of claim 27 wherein the stent is a self-expanding stent.

29. A stent delivery system, comprising:
an introducer catheter having a distal end and a distal portion;
a stent preloaded within the distal portion of the introducer catheter, the stent having a proximal end and a distal end;
a pusher assembly including a soft pusher member configured to urge the preloaded stent from the introducer catheter within which the preloaded stent is slidably disposed, the pusher assembly comprising a first tubular portion and a second tubular portion, at least a part of the second tubular portion extending distally from the first tubular portion, the second tubular portion including a flexible section and a stent-carrying section located distal to the flexible section, the soft pusher member being disposed along the second tubular portion at a location which is either proximal to or within the stent-carrying section, the soft pusher member being made of a low density polymer and configured to cooperate with the preloaded stent for absorbing preload pressure of the preloaded stent when the soft pusher member is positioned at the acute bend during deployment of the stent; and
a distal tip affixed about the distal end of the second tubular portion, the stent being tightly held between the distal tip and the face of the pusher member such that during deflection of the stent introducer apparatus, the point along the introducer catheter that receives the largest amount of bending stress is located proximal the pusher member,
the flexible section of the second tubular portion having a preselected length and a location along the pusher assembly such that when the pusher assembly and the preloaded stent are disposed within the introducer catheter and are subjected to lateral bending stresses at the known general location in the body, the flexible section of the second tubular portion traverses the known general location in the body, whereby the likelihood of a kink occurring in the introducer catheter is greatest within a region corresponding to the region of greatest flexibility of the pusher assembly.

30. A stent delivery system, comprising:
an introducer catheter having a distal end and a distal portion;
a stent preloaded within the distal portion of the introducer catheter, the stent having a proximal end and a distal end;
a pusher assembly slidably disposed within the introducer catheter, the pusher assembly including both a second tubular portion having a first outer diameter and including a distal end; and a first tubular portion having a second outer diameter and located proximal of the second tubular portion, the second outer diameter being greater than the first outer diameter; and
a pusher member comprising a polymer material, the pusher member being located distal to the second tubular portion and in close proximity with the proximal end of the stent to absorb preload pressure of the stent, wherein the likelihood of a kink between the stent and the pusher member is reduced.

31. The stent delivery system of claim 30 wherein the polymer material comprises polytetrafluoroethylene.

32. The stent delivery system of claim 30 wherein the polymer material further includes a radiopaque filler material.

33. The stent delivery system of claim 30 wherein a distal end of the pusher member is in contact with the proximal end of the stent.

34. The stent delivery system of claim 30 wherein a distal end of the pusher member is in contact with the proximal end of the stent and conforms to the proximal end of the stent.

35. A stent delivery system for use in target ducts or vessels having an acute bend at a known general location in the body of a patient, the system comprising:
a pusher assembly including:
a first tubular portion;
a second tubular portion, at least a part of the second tubular portion extending distally from the first tubular portion, the second tubular portion including a flexible section and a stent-carrying section located distal to the flexible section; and
a soft pusher member configured to urge a preloaded stent from an introducer catheter within which the preloaded stent is slidably disposed, the soft pusher member being disposed along the second tubular portion at a location which is either proximal to or within the stent-carrying section, the soft pusher member being made of a low density polymer having a radiopaque filler and configured to cooperate with a preloaded stent for absorbing preload pressure of the preloaded stent when the soft pusher member is positioned at the acute bend,
the flexible section of the second tubular portion having a preselected length and a location along the pusher assembly such that when the pusher assembly and the preloaded stent are disposed within the introducer catheter and are subjected to lateral bending stresses at the known general location in the body, the flexible section of the second tubular portion traverses the known general location in the body, whereby the likelihood of a kink occurring in the introducer catheter is greatest within a region corresponding to the region of greatest flexibility of the pusher assembly.
